(19) United States
(12) Patent Application Publication
(10) Pub. No.: US 2025/0041617 A1
(43) Pub. Date: Feb. 6, 2025

(54) DEVICE FOR SKIN AND FAT TREATMENT

(71) Applicant: LUMENIS BE LTD., Yokneam (IL)

(72) Inventors: AMIKAM GERSHONOWITZ, Tel Aviv (IL); Moshe Elazar, Kadima-Zoran (IL)

(21) Appl. No.: 18/922,848

(22) Filed: Oct. 22, 2024

Related U.S. Application Data
(63) Continuation of application No. PCT/IL2023/050423, filed on Apr. 24, 2023.
(60) Provisional application No. 63/334,836, filed on Apr. 26, 2022.

(51) Int. Cl. A61N 1/40

(52) U.S. Cl. CPC A61N 1/403

(57) ABSTRACT

Devices for tissue treatment and fat removal are provided. In one embodiment, a device for skin tissue treatment is provided comprising: a handpiece comprising a first part configured to be positioned outside a body of a patient in proximity to a tissue portion to be treated, and a second part configured to be inserted into the body of the patient under the skin tissue surface, in proximity to or inside the tissue portion to be treated; and an RF system comprising an RF generator operable for generating RF signal and an electrode arrangement electrically coupled to the RF generator and operable for receiving the RF signal delivering RF energy to the tissue portion to cause at least one of destruction of adipose tissue cells and tissue tightening at the tissue portion, the electrode arrangement is mounted either on the first part or on the second part. In some embodiments, the second part is devoid of energy sources and gets passively heated by comprising a conductive material located inside the RF field. In some embodiments, a temperature indication system provides temperature data indicative of the temperature at the treated tissue portion.

### TECHNICAL FIELD

The present disclosure relates to the field of aesthetic devices. Particularly, but not exclusively, the present disclosure relates to a device for skin treatment, including skin tightening and fat reduction or/and removal.

### BACKGROUND

Adipose tissue, more commonly known as “fat”, is formed of cells containing stored lipid. A single droplet of lipid occupies most of the volume of the cell. An excess of adipose tissue, i.e., obesity, may be unhealthful in that it gives rise to varying health problems in human beings, both on physical and psychological levels. Obesity typically increases the risk of conditions such as heart disease, high blood pressure, osteoarthrosis, bronchitis, hypertension, diabetes, deep-vein thrombosis, pulmonary emboli, varicose veins, gallstones and hernias. In general, the skin tissue of a subject includes an epidermis (outer) layer over a dermis (deeper) layer. Passing through the epidermis layer into the dermis layer are sweat gland ducts and hair follicles. Immediately beneath the dermis layer is the subdermal plexus of small blood vessels. Further, disposed beneath the dermis layer is a layer of varying thickness in different people, of adipose tissue. There are some people in which the adipose tissue layer becomes quite large; sometimes in cases of extreme obesity. It is desirable to eliminate some of the adipose tissue for both cosmetic and fundamental health reasons.

### SUMMARY OF THE DISCLOSURE

Additional features and advantages are realized through the techniques of the present disclosure. Other embodiments and aspects of the disclosure are described in detail herein and are considered a part of the disclosure.

In accordance with a first aspect of the presently disclosed subject matter, there is provided a device for skin tissue treatment comprising:
a handpiece comprising:
a first part configured to be positioned outside a body of a patient in proximity to a tissue portion to be treated,
a second part configured to be inserted into the body of the patient under the skin tissue surface, in proximity to or inside the tissue portion to be treated,
   and
   an RF system comprising an RF generator operable for generating RF signal and an electrode arrangement electrically coupled to the RF generator and operable for receiving the RF signal delivering RF energy to the tissue portion to cause at least one of destruction of adipose tissue cells and tissue tightening at the tissue portion, wherein the electrode arrangement is mounted either on the first part or on the second part.

In some embodiments, the device comprises a controller in communication with the RF system for controlling the RF system to cause the at least one of the destruction of the adipose tissue cells and the tissue tightening at the tissue portion.

In some embodiments, the electrode arrangement is mounted on the first part, and said second part at least partially comprises or is made of an electrically conductive material that when being subject to the RF energy having specific parameters heats up and causes the destruction of the adipose tissue cells in vicinity thereof.

In some embodiments, the electrode arrangement comprises a first sub-arrangement mounted on the first part and operable, when activated, to cause said tissue tightening to a skin surface of the tissue portion, and a second sub-arrangement mounted on the second part and operable, when activated, to cause said destruction of adipose tissue cells at the tissue portion.

In some embodiments, the second part is hollow and comprises a channel extending proximally from a distal end of the second part and configured for extracting liquified fat from the tissue portion.

In some embodiments, the device comprises a temperature indication system configured to provide temperature data indicative of temperature of the tissue portion and/or surroundings of the tissue portion. The temperature indication system may comprise a temperature sensor mounted on the first part or/and the second part. The temperature indication system may comprise a thermal camera configured and operable to provide the temperature data being in a form of visual data, the visual data being also indicative of location of the first part within the tissue portion. The thermal camera may be mounted on the first part or/and on a headset worn by a user. The visual data may be provided on glasses worn by the user.

In some embodiments, the electrode arrangement comprises at least one mono-polar electrode. In some embodiments, the electrode arrangement comprises at least one bi-polar electrode pair. In some embodiments, the electrode arrangement comprises at least one multi-polar electrode group.

In some embodiments, the second part is detachably connectable to the handpiece, thereby enabling removing the second part and applying treatment to a skin surface of the tissue portion using only the first part having the electrode arrangement mounted on the first part.

In accordance with another aspect of the presently disclosed subject matter, there is provided a device for skin tissue treatment comprising:
a handheld probe comprising:
a first part configured to be positioned outside a body of a patient in proximity to a tissue portion to be treated,
a second part configured to be inserted into the body of the patient under the skin tissue surface, in proximity to or inside the tissue portion to be treated,
   and
   an RF system comprising an RF generator operable for generating RF signal and an electrode arrangement electrically coupled to the RF generator and operable for receiving the RF signal delivering RF energy to the tissue portion to cause at least one of destruction of adipose tissue cells and tissue tightening at the tissue portion, wherein the electrode arrangement is mounted on the first part, and wherein said second part at least partially comprises or is made of an electrically conductive material that when being subject to the RF energy having specific parameters heats up and causes the at least one of destruction of adipose tissue cells and tissue tightening at the tissue portion.

In some embodiments, the device comprises a controller in communication with the RF system for controlling the RF system to cause the at least one of the destruction of the adipose tissue cells and the tissue tightening at the tissue portion.

In some embodiments, the second part is hollow and comprises a channel extending proximally from a distal end of the second part and configured for extracting liquified fat from the tissue portion.

In some embodiments, the device comprises a temperature indication system as described above.

In some embodiments, the electrode arrangement comprises at least one mono-polar electrode. In some embodiments, the electrode arrangement comprises at least one bi-polar electrode pair. In some embodiments, the electrode arrangement comprises at least one multi-polar electrode group.

In some embodiments, the second part is detachably connectable to the handpiece, thereby enabling removing the second part and applying treatment to a skin surface of the tissue portion using only the first part having the electrode arrangement mounted on the first part.

In accordance with another aspect of the presently disclosed subject matter, there is provided a device for skin tissue treatment comprising:
a handpiece comprising:
a probe configured to be inserted into the body of the patient under the skin tissue surface, in proximity to or inside a tissue portion to be treated, and
an electrode arrangement,
an RF generator electrically coupled to the electrode arrangement and operable for generating and delivering RF signal to the electrode arrangement for delivering RF energy to a tissue portion in vicinity of the electrode arrangement, to cause at least one of destruction of adipose tissue cells and tissue tightening at the tissue portion,
and
a temperature indication system configured to provide visual data indicative of temperature of the tissue portion and/or surroundings of the tissue portion.

In some embodiments, the temperature indication system is configured as described above.

In some embodiments, the device comprises a first part configured to be positioned outside a body of a patient in proximity to the tissue portion to be treated, the first part carrying the electrode arrangement.

In some embodiments, the probe at least partially comprises or is made of an electrically conductive material that when being subject to the RF energy having specific parameters heats up and causes the destruction of the adipose tissue cells in vicinity thereof.

In some embodiments, the temperature indication system comprises a temperature sensor mounted on the first part.

In some embodiments, the electrode arrangement comprises a first sub-arrangement mounted on the first part and operable, when activated, to cause said tissue tightening to a skin surface of the tissue portion, and a second sub-arrangement mounted on the probe and operable, when activated, to cause said destruction of adipose tissue cells at the tissue portion.

In some embodiments, the probe is hollow and comprises a channel extending proximally from a distal end of the probe and configured for extracting liquified fat from the tissue portion.

In some embodiments, the electrode arrangement comprises at least one mono-polar electrode. In some embodiments, the electrode arrangement comprises at least one bi-polar electrode pair. In some embodiments, the electrode arrangement comprises at least one multi-polar electrode group.

In accordance with another aspect of the presently disclosed subject matter, there is provided a device for skin tissue treatment comprising a housing defining a first end and a second end. The first end and the second end may be integrally connected by a handle portion. The first end further defines a substantially flat surface that may be configured to accommodate one or more electrodes and is structured to be placed on the skin outer surface, i.e. contacting the skin epidermis layer of a subject. The second end is configured to accommodate an RF generator and other components. Further, a portion of the second end is structured to receive a probe. The probe is detachably connectable to the portion of the second end and the probe is configured to be inserted inside a body of the subject at a treatment site/area. In some embodiments, the probe comprises a conductive material but no connection to an energy source. In some embodiments, the probe comprises one or more electrodes for RF energy delivery.

The foregoing summary is illustrative only and is not intended to be in any way limiting. In addition to the illustrative aspects, embodiments, and features described above, further aspects, embodiments, and features will become apparent by reference to the drawings and the following description.

### BRIEF DESCRIPTION OF THE ACCOMPANYING FIGURES

The above-mentioned aspects, other features and advantages of the disclosure will be better understood and will become more apparent by referring to the exemplary embodiments of the disclosure, as illustrated in the accompanying drawings.

FIG. 1A illustrates in a block diagram view, a first non-limiting embodiment of a device for skin tightening and fat removal, in accordance with the present disclosure.

FIG. 1B illustrates in a block diagram view, another non-limiting embodiment of a device for skin tightening and fat removal, in accordance with the present disclosure.

FIG. 1C illustrates in a block diagram view, yet another non-limiting embodiment of a device for skin tightening and fat removal, in accordance with the present disclosure.

FIGS. 2-3 illustrate schematic views of a device for skin tightening and fat removal, in accordance with some non-limiting embodiments of the present disclosure.

FIGS. 4-6 illustrate schematic views of the device with a probe having protrusions, in accordance with some non-limiting embodiments of the present disclosure.

FIGS. 7-9 illustrate schematic perspective views of the device having ring protrusions on the probe, in accordance with non-limiting embodiments of the present disclosure.

FIG. 10 illustrates a schematic view of the device with a display unit integrated to a portion of the device, in accordance with non-limiting embodiments of the present disclosure.

FIGS. 11-12 illustrate schematic perspective views of the device having a probe defined with a web tip, in accordance with a non-limiting embodiment of the present disclosure.

FIG. 13 illustrates a schematic perspective view of the device without a probe, in accordance with a non-limiting embodiment of the disclosure.

The figures depict embodiments of the disclosure for purposes of illustration only. One skilled in the art will readily recognize from the following description that alternative embodiments of the unit illustrated herein may be employed without departing from the principles of the disclosure described herein.

### DETAILED DESCRIPTION

The foregoing description broadly outlines the features and technical advantages of the present disclosure in order that the description of the disclosure that follows may be better understood. Additional features and advantages of the disclosure will be described hereinafter which form the subject of the disclosure. It should be appreciated by those skilled in the art that the conception and specific embodiments disclosed may be readily utilized as a basis for modifying or designing other devices for carrying out the same purposes of the present disclosure. It should also be realized by those skilled in the art that such equivalent constructions do not depart from the scope of the disclosure. It is to be expressly understood, however, that each of the figures is provided for the purpose of illustration and description only and is not intended as a definition of the limits of the present disclosure.

Some embodiments of the present disclosure will be descried referring to the accompanying drawings. While some specific terms of “upper,” “lower,” “below”, “above”, “right,” or “left” and other terms containing these specific terms and directed to a specific direction will be used, the purpose of usage of these terms or words is merely to facilitate understanding of the present invention referring to the drawings. Accordingly, it should be noted that the meanings of these terms or words should not improperly limit the technical scope of the present invention.

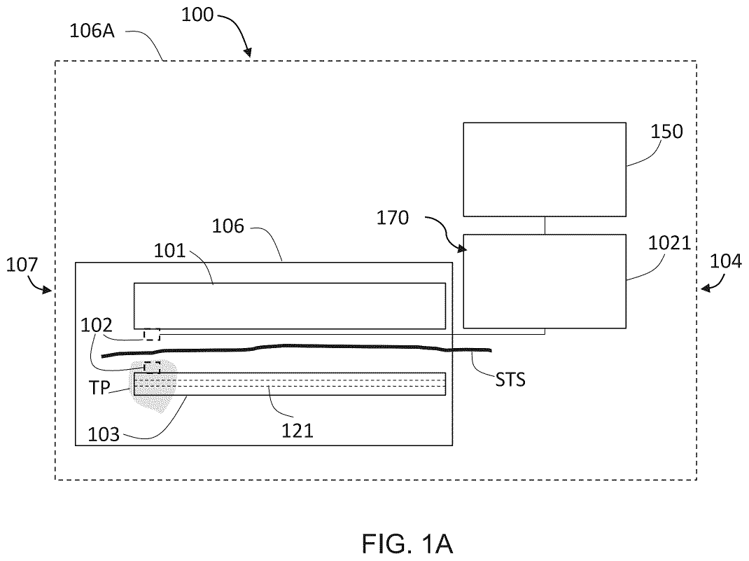

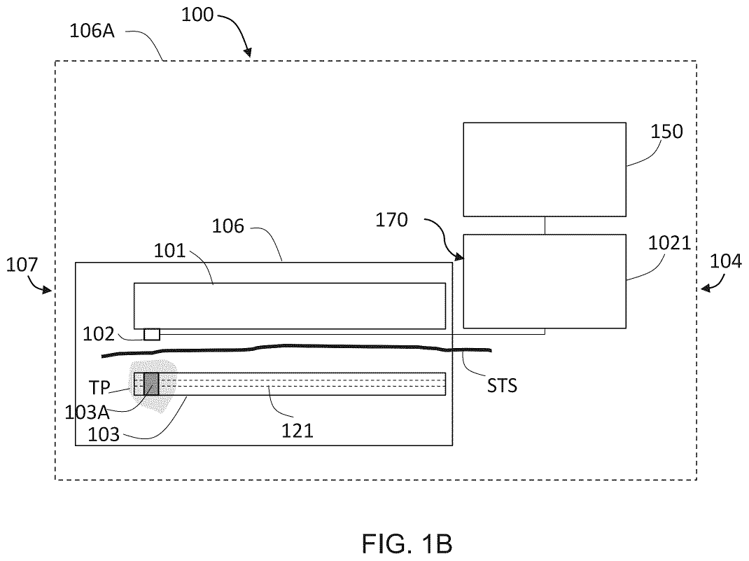

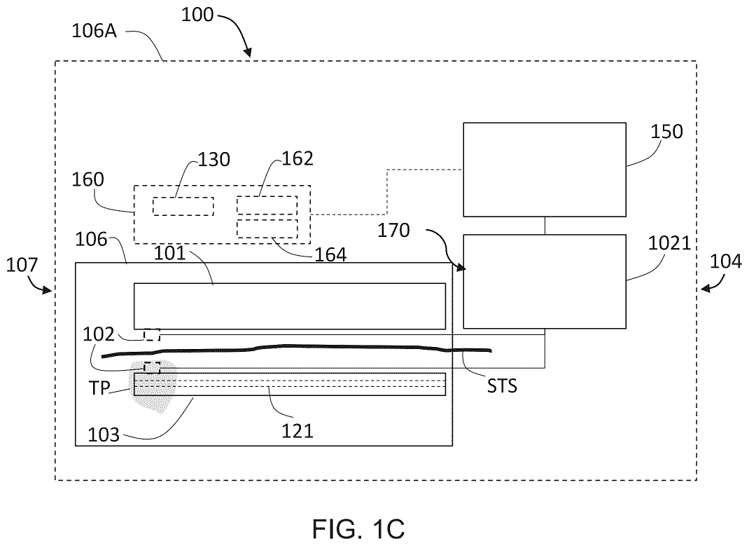

Reference is made to FIG. 1A, illustrating in a way of a block diagram, a non-limiting example of a device 100 for adipose tissue destruction, fat reduction/removal and skin tightening, in accordance with some embodiments of the presently disclosed subject matter. The device 100 includes a first part 101 configured to be positioned outside the body in proximity to the tissue portion TP to be treated, a second part 102 that is configured to be inserted into the body under the skin tissue surface STS in proximity to or inside the tissue portion TP to be treated, a RF system 170 that includes a RF generator 1021 and an electrode arrangement 102 electrically coupled to the RF generator 1021 for delivering RF energy to the tissue portion TP, and a controller 150 in communication with the RF system 170 for controlling the RF system to cause destruction of the adipose tissue cells and/or tissue tightening at the tissue portion. The controller 150 can be a dedicated hardware or a generic hardware running a dedicated firmware or software components, or a combination of both. In one non-limiting example, the controller is a firmware including a set of instructions that can be saved/loaded to a suitable memory or storage device that can be inserted into a dedicated port in the device.

The device 100 is configured as a handheld device having a housing 106 with a distal side/end 107 to be located close to the tissue portion TP and a proximal side 104 located closer to the user. The proximal side 104 of the device may include a handle for easy grasping and manipulation by the user. It is noted that, in some embodiments, the handheld device may have a larger housing 106A that also includes therein the RF generator 1021 and the controller 150. In some other embodiments, one or more of the RF generator 1021 and the controller 150 may be located in a separate enclosure/console and have the necessary wired or wireless communication means with the device and the electrode arrangement 102, as known in the art.

According to the presently disclosed subject matter, as shown with the dashed lines/boxes, the electrode arrangement 102 can be positioned either on a distal side of the first part 101 or on a distal side of the second part 103. The electrode arrangement 102 is configured to receive RF signals from the RF generator 1021 and apply an RF energy field at the tissue portion TP to thereby cause one or both of the following: fat reduction/removal in the adipose layer, and skin tightening at the tissue skin surface and the upper skin layers.

In some embodiments, the electrode arrangement 102 includes two electrode sub-arrangements, a first electrode sub-arrangement located on the first part 101 configured, when activated by the RF generator, to cause skin tightening as mentioned above, and a second electrode sub-arrangement located on the second part 103 configured, when activated by the RF generator independently from the first electrode sub-arrangement, to cause fat reduction/removal, as mentioned above.

In some embodiments, the electrode arrangement 102 is configured as a mono-polar electrode(s) where the RF circuit is closed through another one or more electrodes contacting the body of the person, usually located at the lower side of the person's back. In some embodiments, the electrode arrangement 102 is configured as a bi-polar electrode arrangement where one or more pairs of electrodes are operated in opposite polarities to create corresponding and distinct RF energy fields between each of the one or more pairs. In some embodiments, the electrode arrangement 102 is configured as a multi-polar electrode arrangement where a first group of electrodes is operated at a first polarity and a second group with different number of electrodes is operated at a second opposite polarity to create a plurality of RF energy fields. In some embodiments, the polarity of one or more electrodes in the electrode arrangement may change over time during the treatment, giving a dynamic RF energy field over the treatment site. Any of the above mentioned modes of operation (mono-, bi-, or multi-polar) can be controlled through the controller 150 being programmed accordingly.

The second part 103 has an elongated shaft or cannula shape that is both thin enough and strong enough to be inserted under the skin tissue surface STS into the tissue portion TP at the treatment site. In some embodiments, the second part 103 is hollow and contains a channel 121 extending proximally from the distal end, through which the destroyed/melted fat tissue can be withdrawn out of the body, possibly by communicating with a vacuum source (not explicitly shown) connected to or included in the device 100.

The housing 106 may include one or more buttons for activating and deactivating the electrode arrangement for applying the desired tissue treatment.

Reference is made to FIG. 1B, illustrating in a way of a block diagram, a non-limiting example of the device 100 where the electrode arrangement 102 is located on the first part 101 positioned outside the body, in accordance with some embodiments of the presently disclosed subject matter. As shown, the second part 103 carries thereon or includes a portion 103A that is located at the distal side of the second part, that is inserted into the body under the skin surface, such that this portion 103A is located within the range of the RF energy field applied by the electrode arrangement 102. During treatment, the portion 103A is located in proximity of/inside the tissue portion TP. The portion 103A includes or is made from an electrically conductive material and the remaining part or the whole second part (in case the portion 103A is not an integral part of the second part 103 but is mounted thereon), is made from an electrically non-conductive material. The portion 103A is devoid of any connection to the RF generator. In this configuration, when the electrode arrangement 102 on the first part is operated to apply the RF energy field, the portion 103A or the conductive material included therein heats up and when adequate parameters of the RF energy field are provided to heat the portion 103A to adequate temperature range, heat diffusion from the heated portion 103A causes the heating and destruction of the fat tissue in proximity thereof. In this configuration, the device 100 can be used for both fat removal and skin tightening due to the effect of heating applied on both deep adipose layers surrounding the second part as well as shallow skin layers beneath the external electrode arrangement 102. As will be appreciated from the non-limiting examples detailed below, a plurality of spaced apart conductive regions 103A may be provided on the second part 103.

Reference is made to FIG. 1C, illustrating in a way of a block diagram, a non-limiting example of the device 100 where the electrode arrangement 102 is either located on the first part 101 positioned outside the body or on the second part 103 positioned inside the body, in accordance with some embodiments of the presently disclosed subject matter and as explained above with reference to FIG. 1A. As shown, in this non-limiting example, the device 100 includes a temperature indication system 160 configured and operable to provide information about the temperature of the environment of the treatment site(s). In some embodiments, the temperature indication system 160 provides information about temperature of the skin surface in vicinity of the first part and/or the electrode arrangement mounted thereon. In some embodiments, the temperature indication system 160 provides information about core temperature inside the body in vicinity of the second part and/or the electrode arrangement mounted thereon.

In some embodiments, the temperature indication system 160 provides temperature sensing data of the temperature on the surface or inside the body by way of a temperature sensor 130 mounted on the first part and/or the second part. The temperature indication system 160 provides the temperature sensing data in real time.

In some embodiments, the temperature indication system 160 includes a temperature sensor being a thermal camera 162, e.g. an Infra-Red (IR) camera, having a field of view covering and/or tracking the treatment site, inside or outside the body, and configured to provide the temperature sensing data. In some embodiments, the IR camera can be mounted on the first part. In some embodiments, the IR camera is mounted on a headset being worn by the user. The IR camera allows the user to see the second part inside the body in addition to the treatment site.

In some embodiments, the temperature indication system 160 includes a visual or/and audible data presentation unit 164 such as a display or/and speaker to convey the temperature sensing data to the user. In some embodiments, the data presentation unit 164 is in a form of a display integrated into glasses worn by the user.

In some embodiments, the controller 150 registers over time the temperature sensing data received the temperature sensor/IR camera, to enable the user to track the temperature profile over time or/and over different spots in the treatment site. This enables the user to plan additional treatments, if and as needed, on the treatment site.

In is noted that, in some embodiments and though not explicitly show, the device illustrated in FIG. 1C can additionally include one or more regions 103A as described with reference to FIG. 1B

In the following, non-limiting examples and configurations of the device 100 and the different elements/features are described. It is noted that the following examples should not limit the presently disclosed subject matter and other and different combinations can be envisioned by a person skilled in the art. Additionally, it is appreciated that further and specific details described below with relation to the different elements/features can be also applicable to the corresponding general elements/features described above in FIGS. 1A-1C.

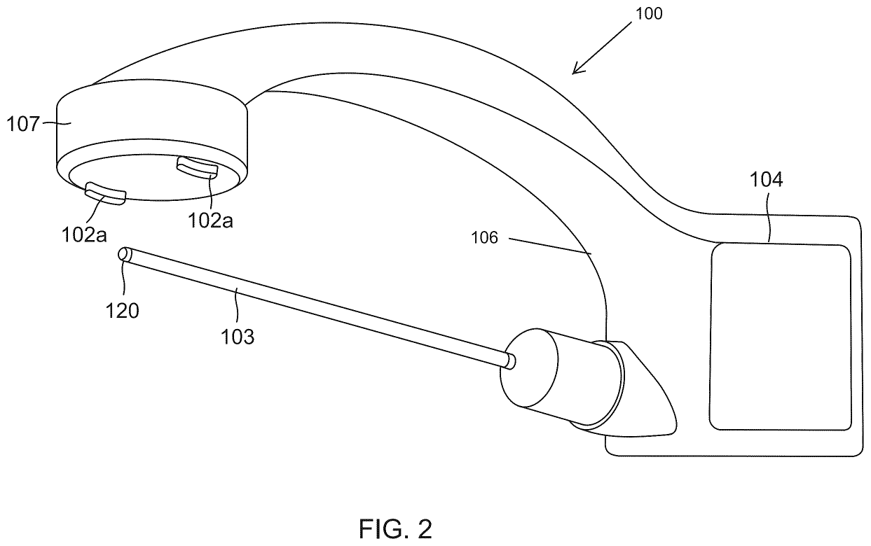

Referring to FIG. 2, the figure illustrates a perspective view of a non-limiting example of a device 100 for adipose tissue destruction, fat reduction/removal and skin tightening, in accordance with the presently disclosed subject matter. As shown, the device 100 includes a housing 106. The housing 106 may be defined with a first, distal, end 107 and a second, proximal, end 104. The first end 107 and the second end 104 may be integrally connected by the first part 101 formed as a handle portion 101. The housing 106 and/or the first part/handle portion 101 may be a hollow structure configured to accommodate one or more components including wiring harness, optical cables and the like passing from the second end 104 to the first end 107. In some embodiments, the housing 106 at the second end 104 may be configured to accommodate at least one of RF source/generator [not shown], a compartment for receiving battery modules [not shown] in case of battery powered or a socket to receive external electrical power supply. Further, the second end 104 may be defined with one or more units and/or ports that may aid in communicatively coupling the device 100 to other equipment.

The first end 107 of the device 100 defines a substantially flat surface that is configured to accommodate the electrode arrangement (one or more electrodes) 102 coupled to the RF source. Portions of the electrodes 102 may protrude downwardly from the substantially flat surface on the first end 107. In some embodiments, as shown in this non-limiting example, two electrodes 102a are provided having opposite polarities to create a bipolar RF field.

In some embodiments, the electrodes 102a are positionable on a skin surface above the treatment area and may be applied to the skin surface. In some embodiments, the size of the electrodes and/or the energy applied thereby are adjusted to create skin tightening without thermal damage of the dermis and epidermis. The first end 107 of the device carrying the electrodes 102 may be designed for smooth movement over the skin creating uniform effect in the skin and the adipose tissue.

Further, the device 100 includes the second part a probe 103, and the second part/probe 103 may be detachably connectable to a portion of the second end 104 of the housing 106. In some embodiments, the probe 103 may be an elongated cylindrical structure such as a shaft or a hollow tube. The probe 103 may include a proximal end at the proximal side 104 of the device and a distal end at the distal side 107 of the device. In some embodiments, the proximal end of the probe 103 may be connectable to the portion of second end 104 of the housing 106. In some embodiments, the proximal end of the probe 103 may be configured with attaching structure to the device including but not limited to magnetic structure, to attach to the second end 104 of the housing 106. The probe 103 is configured to be inserted into a body of the person, i.e., in the vicinity of the adipose tissue of the person.

In some embodiments, the probe 103 is made from a conductive material absent any energy source connection. In some embodiments, the probe 103 is a made from a non-conducting material including one or more portions 120 (region 103A) made from a conductive material. By way of specific example, when the probe (or any portion of the probe) includes a conductive material without any energy source attached, the electrodes 102 are connected to RF energy, the probe is passively heated by the RF field generated by electrodes 102 such that the probe is configured for fat destruction. When the electrodes 102a are set up in the device as a bipolar RF energy field passing from one electrode to the other, the distal end of probe 103 which is exposed to the RF field, is heated, and as a result may be configured for the destruction of the adipose tissue in the vicinity of the distal end of the probe 103.

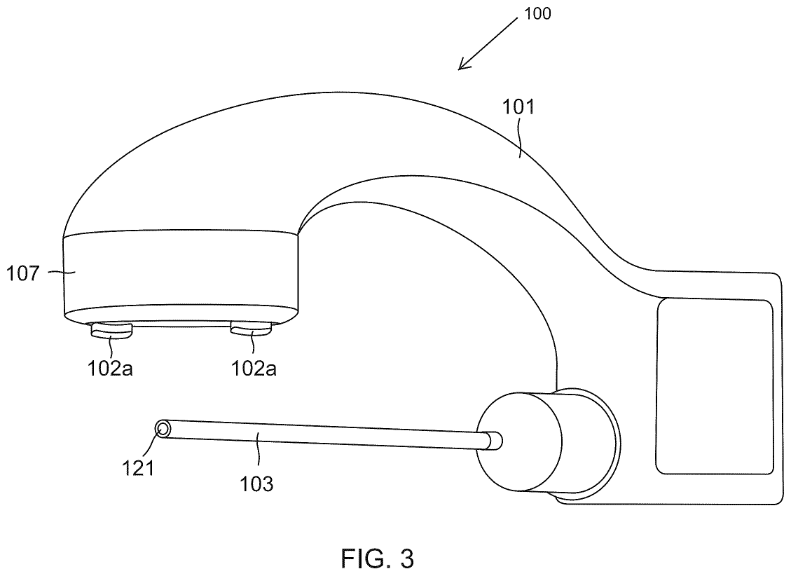

Referring now to FIG. 3, in some non-limiting embodiments, the probe 103 is hollow having a channel (121, as shown in FIG. 1A) defined thereof and may be referred to as a cannula. When the probe includes a cannula, the distal end of the probe is open and includes an aperture 121, and the cannula is configured to evacuate liquified fat from the treated patient.

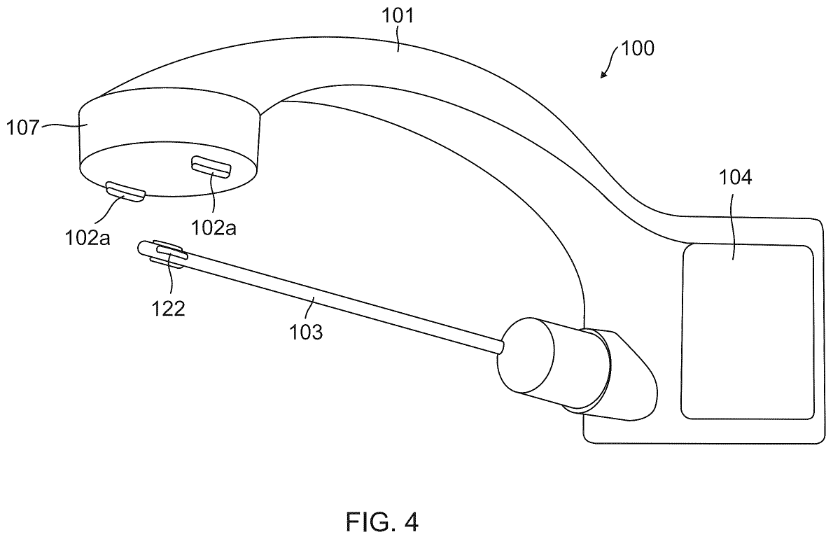

FIG. 4 illustrates a probe 103 including protrusions 122. In some embodiments, these protrusions 122 are four RF probe electrodes connected to an energy source where two protrusions are one polarity and two protrusions are a second opposite polarity. In some embodiments, there may be any number of protrusions and may work in different electrode RF systems or modes, such as bipolar pairs or multipolar configuration. In some embodiments, the polarity of each electrode may change over time and during the treatment, giving a dynamic RF field over the treated area. In some embodiments, the probe in FIG. 4 is made of a non-conducting material and the protrusions 122 are conductive material absent any energy source. As discussed above, conducting elements, such as protrusions 122, when exposed to an RF field will be heated and may be configured for the destruction of the adipose tissue in the vicinity of the distal end of the probe. In some embodiments, protrusions 122 can be a predefined mixture/combination of RF electrodes and conductive elements absent any energy source.

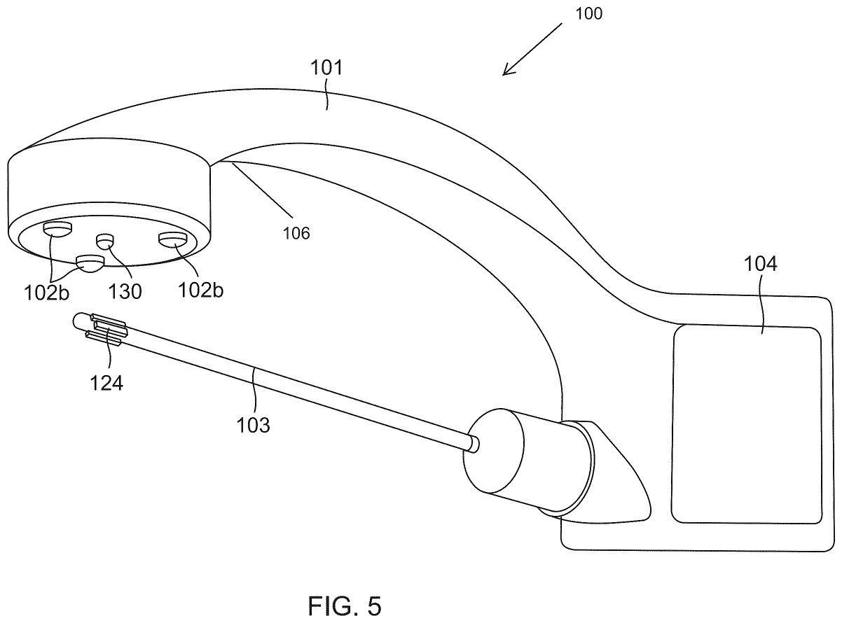

FIG. 5 illustrates, in some non-limiting embodiments, electrodes 102b on the flat surface of first end 107 which is configured to accommodate three electrodes 102b, also known as a tripolar arrangement. In some embodiments, when a tripolar arrangement is used, two electrodes are operated with one polarity of RF energy and the third is operated with an opposite polarity. In some embodiments, any odd number of electrodes may be used such that an even number of electrodes are one polarity and an odd number are the opposite polarity. FIG. 5 further illustrates that, in some embodiments, a temperature indication system, such as a temperature sensor 130 or/and an IR camera (not shown), may be placed on the surface of first end 107, e.g. on the flat surface carrying the electrodes. The probe 103 may further include protrusions 124 of a different shape than FIG. 4. In some embodiments, protrusions of the probe 103 are any shape. In some embodiments, the protrusions 124 are four RF probe electrodes connected to an energy source where two protrusions are one polarity and the other two protrusions are a second opposite polarity. In some embodiments, the probe in FIG. 5 is a non-conducting material and the protrusions 124 are conductive material absent any energy source and the protrusions get heated being located within the generated RF field as described above.

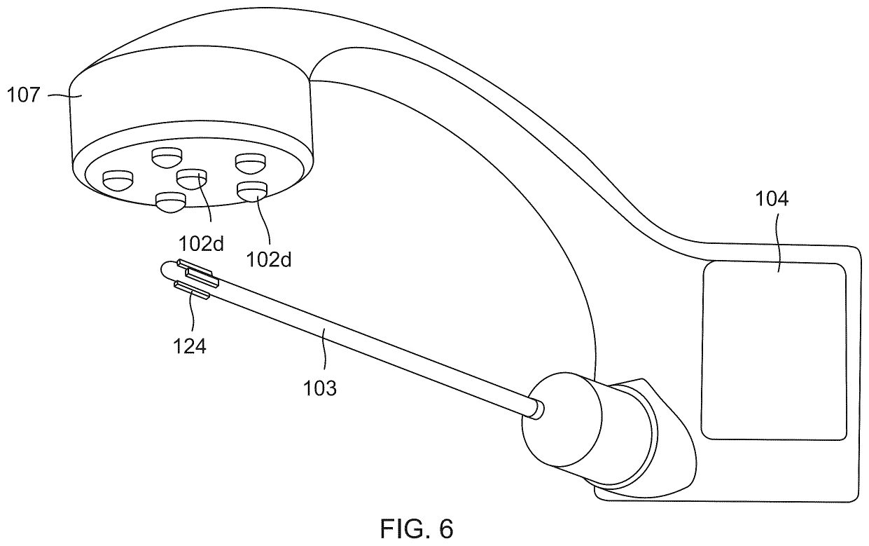

Referring now to FIG. 6 which illustrates that, in some non-limiting embodiments, there are multiple electrodes 102d of even number configured such that half of electrodes are one polarity and the other half of electrodes are a second opposite polarity. Again, the protrusions 124 can be partially RF electrodes, partially conductive elements or/and a combination thereof.

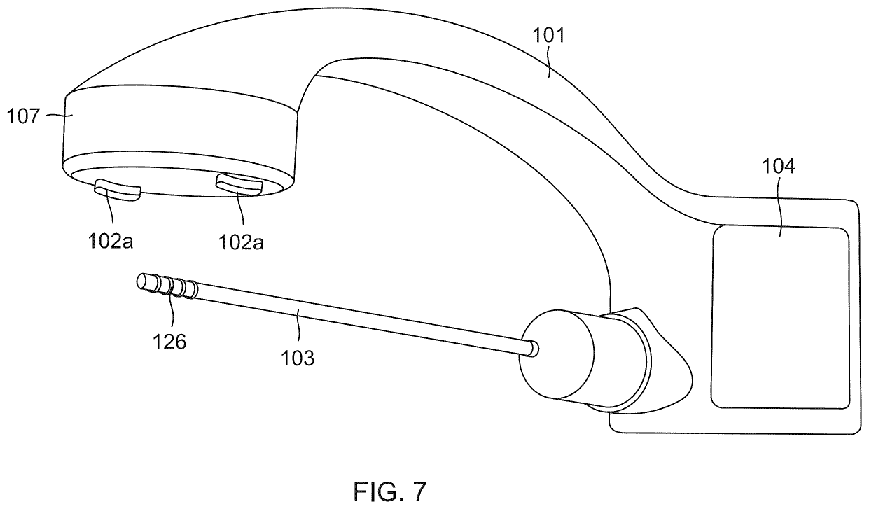

FIG. 7 illustrates the probe 103 including ring protrusions 126. In some embodiments, these ring protrusions 126 are even number (e.g., four) of RF electrodes connected to an energy source where half (two) of the ring protrusions are one polarity and the other half (two) protrusions are a second opposite polarity. Alternatively, ring electrodes may work in a multipolar mode and the polarity of each electrode may change over time and during treatment. In some embodiments, the polarity of each electrode in any mode may change over time and during use. In some embodiments, the probe 103 of FIG. 7 is made of a non-conducting material and the ring protrusions 126 are conductive material absent any energy source and the ring protrusions get heated being located within the RF field as described above.

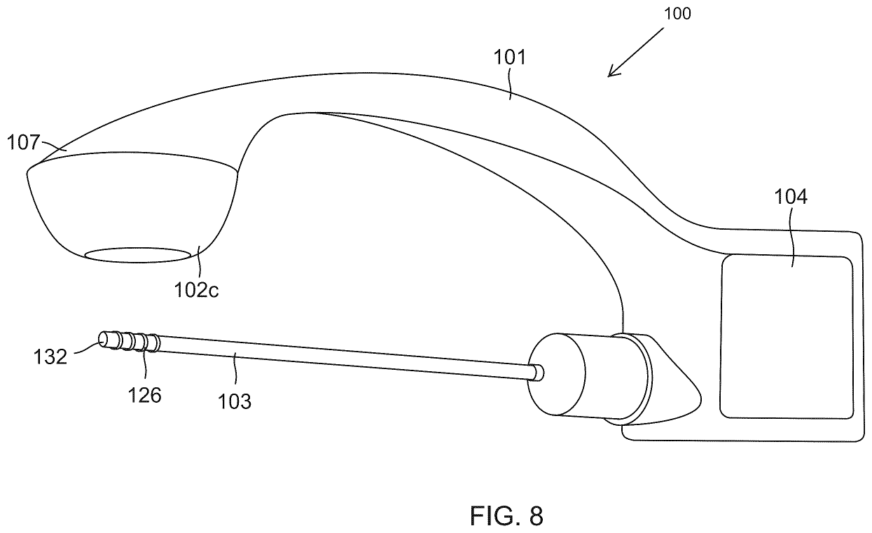

FIG. 8 illustrates that, in some non-limiting embodiments, the first end 107 of the first part of the device includes a monopolar electrode 102c. The RF signal will be then closed via another electrode not located on the device but which is placed in contact with the body of the person. In some embodiments, the probe 103 with ring protrusions 126 may further include an aperture 132 (as described in FIG. 3) such that the probe may also be used as a cannula to withdraw the fat outside the body.

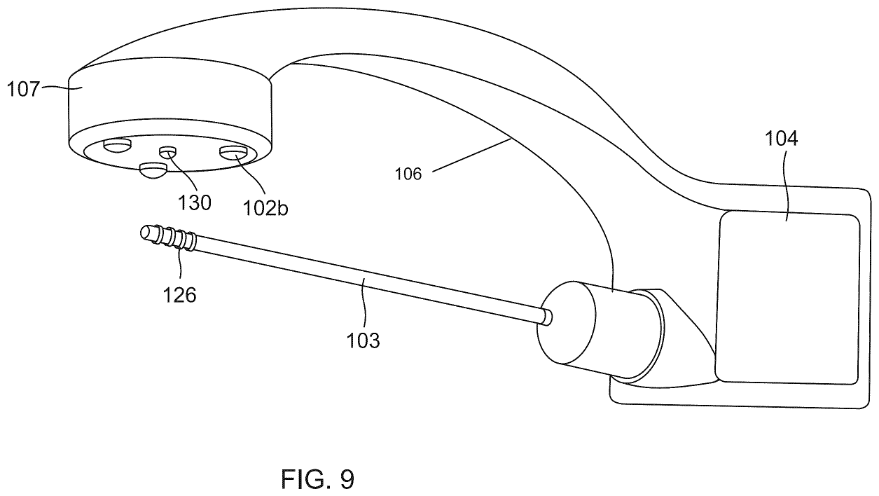

FIG. 9 illustrates that, in some non-limiting embodiments, there may be a tripolar electrode set up 102b with both a temperature sensor 130 (or/and IR camera) and a probe with ring protrusions 126. Alternatively or additionally, in some embodiments, the probe 103 includes a temperature sensor configured to sense temperature of the skin tissue surrounding the probe, e.g. an IR camera mounted externally to the body or a temperature sensor mounted on the probe 103.

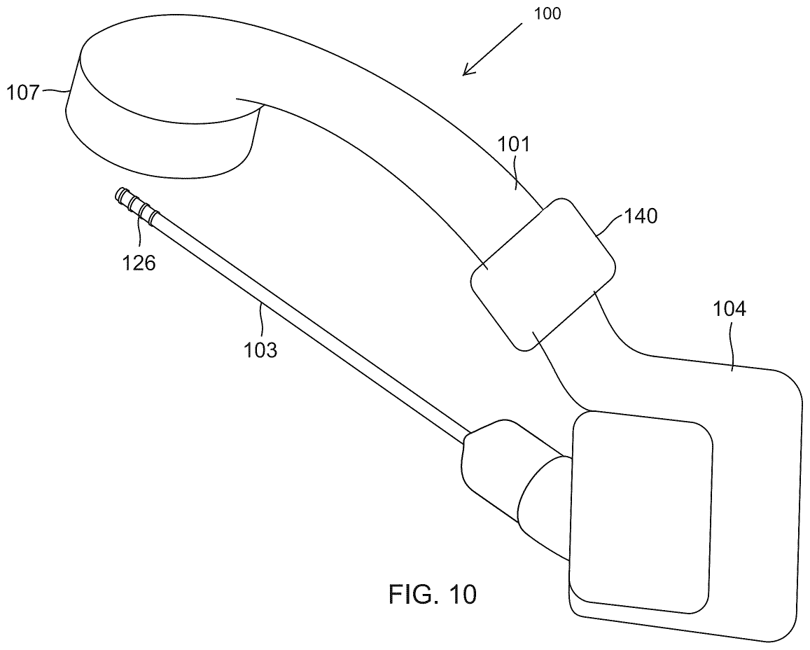

Referring now to FIG. 10, this figure illustrates that, in some embodiments, the device further comprises a view screen 140 (data presentation unit) configured to display temperature sensor readings for a temperature sensor on the probe or a temperature sensor placed on the flat surface of first part, or both.

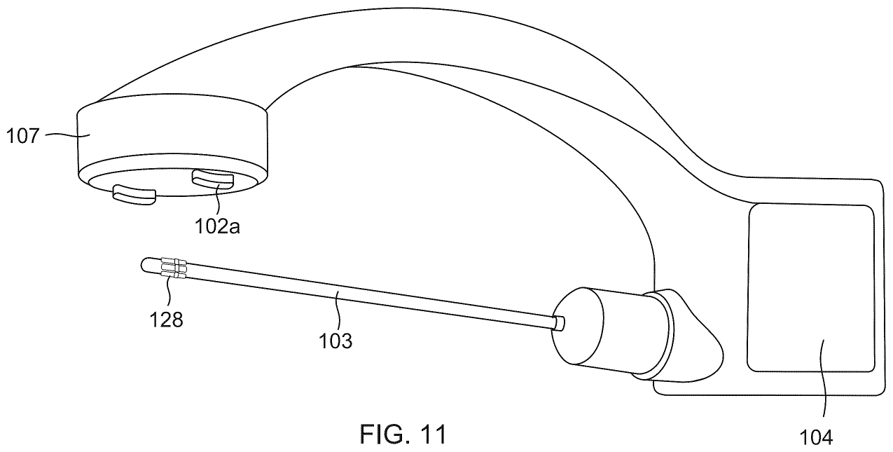

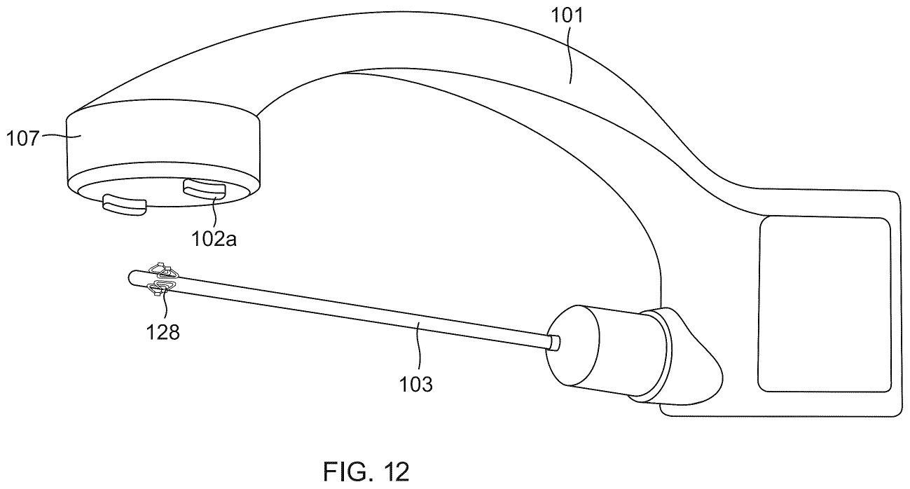

In some embodiments, the distal end of the probe 103, as seen in FIGS. 11 and 12, includes a web tip 128. The web tip is configured to operate between a closed position as shown in FIG. 10 and an open position as shown in FIG. 11. In some embodiments, the web tip 128 includes a conductive material absent an energy source, such that it gets heated when exposed to the RF energy within the RF field, and the probe 103 is made from a non-conductive material.

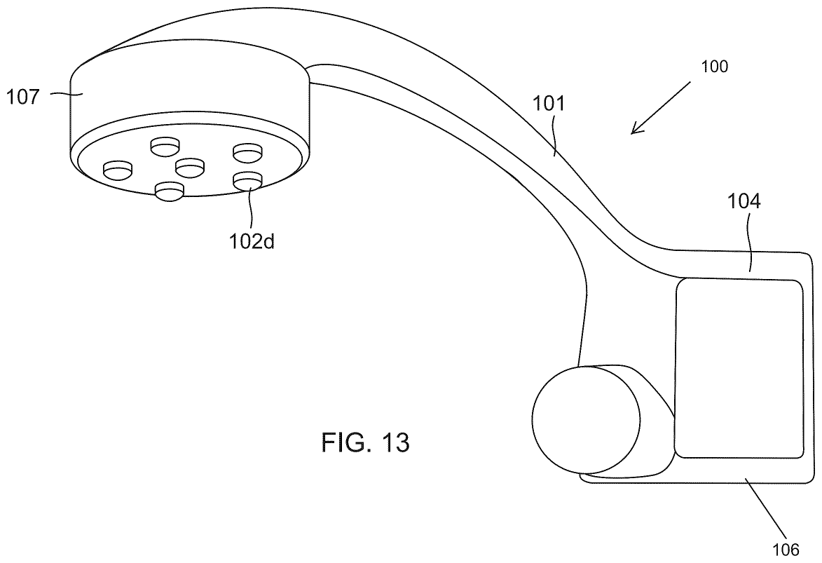

In some embodiments, the device 100 is configured to perform external treatment, i.e., on skin without the aid of the probe 103, as seen in FIG. 13. For example, a detachably connectable probe (second part) can be removed to facilitate treatment by the electrode arrangement 102d mounted on the flat surface of the first part (the handle).

In some embodiments, the device and specifically the probe 103 may be made of biocompatible materials. The term bio-compatible used refers to property of a material being compatible with living tissue. Biocompatible materials do not produce a toxic or immunological response when exposed to the body or bodily fluids. In some embodiments, when RF energy is delivered to electrodes on the device, the RF energy may be delivered at a sufficient level to destroy adipose tissue in the vicinity of the tip or distal end of the probe. In some embodiments, the electrodes 102, when mounted on the first part of the device, may also heat the epidermis layer of the subject below to a sub-necrotic level.

The device may have a probe not connected to an energy source and may be inserted into the adipose tissue. Alternatively, the device may further provide bi-polar, tri-polar or multi-polar RF system on the flat surface of first part and have one or more treatment electrodes on the second part (probe) that is inserted into the adipose tissue.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprises”, “comprising”, or any other variations thereof, are intended to cover a non-exclusive inclusion, such that a mechanism that comprises a list of components does not include only those components but may include other components not expressly listed or inherent to such mechanism. In other words, one or more elements in the device or mechanism preceded by “comprises . . . a” does not, without more constraints, preclude the existence of other elements or additional elements in the mechanism.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

While various aspects and embodiments have been disclosed herein, other aspects and embodiments will be apparent to those skilled in the art. The various aspects and embodiments disclosed herein are for purposes of illustration and are not intended to be limiting.

1. A device for skin tissue treatment comprising: a handpiece comprising:
a first part configured to be positioned outside a body of a patient in proximity to a tissue portion to be treated,
a second part configured to be inserted into the body of the patient under the skin tissue surface, in proximity to or inside the tissue portion to be treated,
a radio frequency (RF) system comprising an RF generator operable for generating RF signal and an electrode arrangement mounted on the first part electrically coupled to the RF generator and operable for receiving the RF signal and delivering RF energy to the tissue portion, and
wherein the second part at least partially comprises or is made of an electrically conductive material that when being subject to the RF energy having specific parameters heats up and causes at least one of destruction of adipose tissue cells and tissue tightening at the tissue portion in vicinity thereof.

2. The device according to claim 1, comprising a controller in communication with the RF system for controlling the RF system to cause the at least one of the destruction of the adipose tissue cells and the tissue tightening at the tissue portion.

3. The device according to claim 1, wherein said second part is hollow and comprises a channel extending proximally from a distal end of the second part and configured for extracting liquified fat from the tissue portion.

4. The device according to claim 1, comprising a temperature indication system configured to provide temperature data indicative of temperature of the tissue portion and/or surroundings of the tissue portion.

5. The device according to claim 4, wherein said temperature indication system comprises a temperature sensor mounted on at least one of: the first part or the second part.

6. The device according to claim 4, wherein said temperature indication system comprises a thermal camera configured and operable to provide the temperature data being in a form of visual data, the visual data being also indicative of location of the first part within the tissue portion.

7. The device according to claim 6, wherein said thermal camera is mounted on at least one of: the first part or a headset worn by a user.

8. The device according to claim 6, wherein said visual data is provided on glasses worn by the user.

9. The device according to claim 1, wherein said electrode arrangement comprises at least one mono-polar electrode.

10. The device according to claim 1, wherein said electrode arrangement comprises at least one bi-polar electrode pair.

11. The device according to claim 1, wherein said electrode arrangement comprises at least one multi-polar electrode group.

12. The device according to claim 1, wherein said second part is detachably connectable to the handpiece, thereby enabling removing the second part and applying treatment to a skin surface of the tissue portion using only the first part having the electrode arrangement mounted on the first part.

13. A device for skin tissue treatment comprising: a handheld probe comprising:
a first part configured to be positioned outside a body of a patient in proximity to a tissue portion to be treated,
a second part configured to be inserted into the body of the patient under the skin tissue surface, in proximity to or inside the tissue portion to be treated,
and
a radio frequency (RF) system comprising an RF generator operable for generating RF signal and an electrode arrangement mounted on the first part electrically coupled to the RF generator and operable for receiving the RF signal and delivering RF energy to the tissue portion to cause at least one of destruction of adipose tissue cells and tissue tightening at the tissue portion, wherein said second part is not electrically coupled to the RF generator and at least partially comprises or is made of an electrically conductive material that when being subject to the RF energy having specific parameters heats up and causes the at least one of destruction of adipose tissue cells and tissue tightening at the tissue portion.

14. The device according to claim 13, comprising a temperature indication system configured to provide temperature data indicative of temperature of the tissue portion and/or surroundings of the tissue portion.

15. The device according to claim 14, wherein said temperature indication system comprises a temperature sensor mounted on at least one of: the first part or the second part.

16. The device according to claim 14, wherein said temperature indication system comprises a thermal camera configured and operable to provide the temperature data being in a form of visual data, the visual data being also indicative of location of the first part within the tissue portion.

17. The device according to claim 13, wherein said electrode arrangement comprises at least one mono-polar electrode.

18. The device according to claim 13, wherein said electrode arrangement comprises at least one multi-polar electrode group.

19. The device according to claim 13, wherein said second part is detachably connectable to the handpiece, thereby enabling removing the second part and applying treatment to a skin surface of the tissue portion using only the first part having the electrode arrangement mounted on the first part.
